(19) United States
(12) Patent Application Publication
(10) Pub. No.: US 2009/0247807 A1
(43) Pub. Date: Oct. 1, 2009

(54) RADIOTHERAPY DEVICE AND METHOD

(75) Inventors: Stephane Lucas, Suarlee (BE); Vincent Nuttens, Namur (BE)

Correspondence Address: MERCHANT & GOULD PC, P.O. BOX 2903, MINNEAPOLIS, MN, 55402-0903, US

(73) Assignee: Facultes Universitaires Notre-Dame de la Paix, Namur (BE)

(21) Appl. No.: 11/721,947

(22) PCT Filed: Dec. 19, 2005
(86) PCT No.: PCT/BE05/00187

§ 371 Date: Jun. 15, 2007

Related U.S. Application Data
(60) Provisional application No. 60/637,480, filed on Dec. 17, 2004.

(51) Int. Cl. A61M 36/12

(52) U.S. Cl. 600/8

(57) ABSTRACT

A radioactive implant for brachytherapy includes a composite radioactive source having at least two different types of radionuclides.

### OBJECT OF THE INVENTION

The present invention is related to the field of treatment by radiations and concerns in particular the use of radioactive sources, adapted for irradiating of cells of the human or animal body.

### STATE OF THE ART

Brachytherapy is a generic term that covers therapeutic treatments inducing the placing of a radioactive source that emits radiations inside the human body. The implant of such a source can be either of a permanent or of a non-permanent nature. For non-permanent implants devices that permit the delivery of a high dose (HDR—high dose rate) are usually used, while permanent implants permit the delivery of a lower dose, usually called “LDR”—low dose rate. If necessary, the delivered dose can also be fragmented in that case.

To have real effectiveness, these implants are disposed within or in the vicinity of the tumour or within or in the vicinity of the volume of infected tissue. These implants appear usually as seeds of rice (seeds) with a length of a couple of millimetres and an internal diameter lower than 1 millimetre. They are traditionally obtained from a biocompatible material, preferably sealed, which is used as a capsule in which a radioactive source is enclosed.

For some time now, there have been suggestions to use windings (springs) made of a wire in an alloy comprising radionuclides for this kind of radiotherapy devices.

The nature of the coating material that forms the capsule or the metal wire that permits winding is such that is must be biocompatible, not toxic for the cells in contact with the implant and if possible not subject to phenomena of opsonisation.

Secondarily, it was proposed to link various of these radioactive devices to one another, a chain or train of different implants linked by for instance a biodegradable material. This would in particular allow an easier localisation of the implants a quick and easy retreat.

Two large families of treatment are considered for the use of brachytherapy. On the one hand the sterilisation of cancer cells or tissues in case of confined tumours, like for instance in the cases of breast cancer, brain cancer, liver cancer, ovary cancer or prostate cancer.

Another envisaged treatment family allows for the use of brachytherapy for the elimination of possibly healthy cells, in the case of stenosis or necroses of the biological canals such as the coronary arteries.

Traditionally, the emitted radiations come from one sole type of radionuclides chosen according to the affected area. Among those most often used are ¹²⁵I, ¹⁰³Pd, ⁹⁰Y, ³²P, ¹⁹²Ir, . . . or also ²¹³Bi, nowadays used in an experimental phase. Each radionuclide has a typical radiation: a low energy photon (¹⁰³Pd), a middle energy photon (¹²⁵I), a high energy photon (¹⁹²Ir), a particle with the same mass as an electron (beta radiation (⁹⁰Y or ³²p) or Auger electron), and an alpha particle (²¹⁶Bi).

The efficiency of the treatment by the different radiations depends on a physical quantity called LET (Linear Energy Transfer). The latter indicates the rate of loss of radiation energy in a material like, for instance, the human body. It is low for a photonic and beta radiation (photon of 4 MeV: 0.3 keV/μm, β de 1 MeV: 0.12 keV/μm) and very high for an alpha radiation (a of 1 MeV: 50 keV/μm).

When the LET increases, the biological efficiency of the radiations on the mutation and the death of cells increases accordingly.

While the alpha particles, beta particles and Auger electrons are hardly penetrating, the photonic radiations penetrate relatively profoundly, depending on their energy. Thus, the use of a low photonic radiation or particle energy facilitates shielding the source, the preservation of healthy tissues and protection of the hospital employees, but has the drawback of limiting the effective volume of the treatment, which can only be compensated by longer exposure (HDR) or by the use of a significant number of implants (LDR). On the other hand, the use of radionuclides that emit very penetrating radiations has the advantage of treating a significant volume, however, it also affects healthy tissues and hospital staff and the radiations cannot be shielded easily.

Depending on what kind of radionuclide is used, the decay time can be as short as a few seconds or as long as a few weeks or even years. It is usually assumed that radionuclides with a short half-life are the more suitable for aggressive tumours and that those with a longer half-life are more apt for less aggressive tumours or profound treatments.

Traditionally, these radio nuclides are integrated in a source that can appear the form of an ink (dried up liquid). They can be present in an ion exchange resin, in a mixture made out of a gel ora powder, in zeolites, or even absorbed on the surfaces of activated particles or graphite marbles or others.

In the particular cases of breast or prostate cancer, permanent brachytherapy devices, comprising either ¹⁰³Pd or ¹²⁵I, are mostly used. ¹⁰³Pd emits XR photons of low energy with a dose distribution that decreases quickly, while ¹²⁵I presents a slightly higher energy with a smoother dose distribution.

In practice, it is thus observed that the number of implants or of brachytherapy elements used for instance for curing breast cancer, will be higher when the radionuclide ¹⁰³Pd is used than when ¹²⁵I is used. It is also observed that the half-life of ¹⁰³Pd is 16,991 days, whereas the one of ¹²⁵I is 59,40 days. Thus, ¹⁰³Pd will preferably be used in case of an aggressive tumour, whereas ¹²⁵I, will be used for a less aggressive tumour.

Furthermore, when ¹⁰³Pd is used the presence of “cold points” is observed on which the minimum distributed dose to eliminate the cancer cells is not delivered; this will particularly happen in case of a shifting of sticks inside the target volume.

In case ¹²⁵I is used, it is observed that the dose distribution, notwithstanding its decrease, does not reach a zero value on longer distances, which in reality means that healthy organs surrounding the cancer tumours could receive a radiation dose that is not zero, which in terms of medical risk leads to serious complications.

Nowadays, it is therefore possible to consider combining several different types of devices to treat a cancer, which allows for combining the advantages of the different treatments and possibly for reducing the disadvantages.

According to particular protocols, is could be possible alternating radioactive brachytherapy devices of ¹⁰³ Pd on the one hand and of ¹²⁵I on the other hand can be considered. It is however observed that the complete elimination of cold points will not be achieved and that the irradiation of healthy tissues of a non-negligible extent will possibly still appear.

### AIMS OF THE INVENTION

The present invention aims at providing an improved solution in comparison to the solutions of the state of the art, and allows a reduction of their inconveniences.

The present invention aims in particular at the possibility to combine the advantages different types of radionuclides offer.

The present invention aims at allowing the combination of different nuclides for one and the same therapeutic application, particularly in the cases of breast and prostate cancer.

The present invention more particularly aims at offering the possibility to play at different types of energy, different lifetimes or different radiations for one and the same therapeutic treatment.

The present invention aims furthermore at offering a solution that allows for the visualization and therefore the localisation of brachytherapy sticks.

### DESCRIPTION OF THE MAIN CHARACTERISTICS OF THE INVENTION

The object of the invention is aimed at combining at least two different types of radionuclides within one single radioactive device made of an implant, in order to realise a composite source, regardless of the geometry or presentation of said radioactive device. This device may also be a product that comprising a solid, adequate and transparent pharmaceutical carrier with radioactive radiation that incorporates the two types of radionuclides. This product can be used as medicament. The object of the invention also concerns the use of this product for the preparation of a medicament for the treatment or prevention of prostate, breast, liver and/or brain tumours. The object of the invention also concerns the use of said product for the preparation of a medicament for eliminating cells and/or tissues, in particular cells or tissues affected by stenosis and/or necroses, in particular in the coronary arteries.

This device or product can be presented in the form of a stick, an element in a chain of sticks, an extendible stick, a metallic or plastic catheter, a wire, a clip, a spiral, a plate . . . .

By type or kind is meant radioactive nuclides of the same chemical nature (same number of Z protons) and of the same atomic mass (A), as well as derived products resulting from the disintegration (e.g.: ¹⁰³Pd*−>¹⁰³Rh+Gamma+XR, ¹⁰³Pd* and ¹⁰³Rh represent the same type of radionuclide). A radionuclide is defined as being a radioactive atom characterised by its number of Z protons and its number of A-Z neutrons.

Preferably, these two types of radionuclides are no radio isotopes, which means that they represent different chemical natures (different Z for the two types). A radio isotope is defined by a radioactive isotope of a very particular element, e.g. odine presents two radio isotopes, ¹²⁵I and ¹³¹I, that is, radio isotopes have a different atomic mass (A) but an identical number of protons (Z).

This device can be either permanently or non-permanently implanted in the human body.

Among the list of radionuclides present in the same medical device, the following non-exhaustive list of radionuclides can be cited: ¹⁴C, ³²P, ³³P, ³⁵S, ³⁶Cl, ⁵¹Cr, ⁵⁵Co, ⁶⁰Co, ⁶³Ni, ⁶⁴Cu, ⁶⁷Cu, ⁶⁸Ge, ⁹⁰Y, ⁸⁹Zr, ⁹⁹Mo, ^{99/99m}Tc, ¹⁰³Pd, ¹¹²Pd, ¹¹⁰Ag, ¹¹²Ag, ¹¹³Ag, ¹¹¹In, ¹²³I, ¹²⁴I, ¹²⁵I, ¹³¹I, ¹³³Xe, ¹³¹Cs, ¹³⁷Cs, ¹⁴² Pm, ¹⁵³Gd, ¹⁵⁹Gd, ¹⁶⁶Ho, ¹⁶⁹Yb, ¹⁸¹W, ¹⁸⁶Re, ¹⁸⁸Re, ¹⁹²Ir, ¹⁹⁴Ir, ¹⁹⁸Au, ¹⁹⁹Au, ²¹⁶Bi, ²¹¹At, ²⁴¹Am (or another equivalent radioactive element).

Radiation means either a radiation of a particular type (such as an α, β, Auger electron or neutron radiation), or a radiation of a wave undulating type (γ or XR radiation).

Depending on the application in which the radionuclides will be used, different configurations of combinations of radioactive elements will be considered within the same device:
Use of various types of radionuclides to obtain different types of radiations. To accomplish this, radionuclides emitting radiations of different types (XR, Beta, Gamma, Alpha . . . ) are put together and encapsulated. By varying their concentrations within the device, a regulation is obtained that allows supporting one radiation (for instance Beta) relative to another (e.g. Auger). When also used for aims other than brachytherapy, this configuration allows combining a radiation type used for diagnostics aims (e.g. ^{99m}Tc) and a radiation type used for therapeutic aims (e.g. ²¹¹At). By doing this, it is possible to track the regression of tumours in real time.
Use of various types of radionuclides for a same type of radiation, but with different energies. Radionuclides emitting radiations of the same type (XR, Beta or Auger) but with different energies are then put together and encapsulated. By varying their concentrations within the device, a regulation is obtained that allows supporting for instance a low energy radiation relative to a high energy radiation. When used in nuclear medicament, this configuration allows treating dispersed tumours effectively and homogeneously. In this respect the combination of ⁹⁰Y (Beta radiation with an average energy of 934 keV) and ¹¹⁹Au (Beta radiation with an average energy of 115 keV) can for instance be mentioned.
Use of different types of radionuclides with different half-life times.
When used in nuclear medicament, a radionuclide with a short half-life “boosts” the treatment and can be mixed with a radionuclide with a longer half-life, used by way of basic treatment. This modulation must, of course, be studied as to the radio sensibility of the cells. In this respect, a combination of ¹⁰³Pd (half-life of 17 days) with ¹⁸¹W (half-life of 121 days) could be considered.
Use of different types of material, among which are one or more types of radionuclides with contrast agents. These contrast agents can be magnetic materials, such as Fe, Gd and Ga oxides, which enable the use in magnetic resonance imaging (MRI) so as to allow localising brachytherapy elements.
Use of different types of materials, among which are one or more types of radionuclides mixed with non-radioactive elements, such as ¹⁰²Pd, ¹⁰³Rh or elements that will be selected in view of their ability to generate additional XR photons by fluorescence.
Use of different types of materials, among which are one or more types of radionuclides with microwave absorbing materials, in order to produce heat, so as to combine a treatment by irradiation and a treatment by hyperthermia.

Traditionally, radionuclides and/or combined elements are included within one and the same source, which is preferably solid, presented in the form of for instance an ink, within an ion exchange resin, in a mixture made of a gel, or within a powder mixture in a polymeric coating, in zeolites or even absorbed at the surface of activated particles or graphite marbles, etc.

According to another embodiment, a liquid or gas source could be considered.

In a last possibility would be their presence within nanostructures or microstructures coated by a carbon layer.

Advantageously, the capsule that forms the external coat of the brachytherapy element (radioactive device) (or pharmaceutical carrier of the product of the invention) is made of a biocompatible, possibly biodegradable material. Preferably, this material is a polymeric material, such as phenyletheretherketone (PEEK), nylon or polyurethane. Of course, other materials or metallic materials such as a titan coating, a carbon coating, etc. could also be considered.

In a particularly advantageous way, the internal activity fractions for two radionuclides of different types are between the range of 0.01/99.99% and 99.99/0.01%.

It is meant by the internal activity fraction of a radionuclide included in a source with several radionuclides, the ratio of internal activity of the considered radionuclide towards the sum of the internal activities of the radionuclides present in the source. The term internal activity specifies that the activity in terms of the number of disintegrations per second of the radioactive source is concerned.

According to a particularly preferred embodiment of the invention, a mixture of radionuclides radionuclides ¹⁰³Pd/¹²⁵I is combined in the form of an ink present in a capsule of biocomposite material.

Preferably, the internal activity fractions for the couple ¹⁰³Pd/¹²⁵I are comprised within a ratio that is in its turn comprised between the range of 60/40% to 90/10%, and are preferably near a ratio of 75/25%.

### SHORT DESCRIPTION OF THE FIGURES

FIGS. 1, 2, 3 and 4 represent different embodiments of capsules or other supports that implement the principle of the present invention.

FIG. 5 represents the distribution of a radial dose in relation to the radiation distance, for the two types of radionuclides most often used in case of prostate cancer, namely ¹⁰³Pd and ¹²⁵I.

### DESCRIPTION OF SEVERAL PREFERABLE EMBODIMENTS OF THE PRESENT INVENTION

The present invention concerns the association of at least two types of radionuclides within one and the same source, that is thus a composite source incorporated in a brachytherapy element. This combination not only allows recovering the advantages typical of each one of both radionuclides, but obtaining a synergetic effect in the treatment of cancers or apoptosis (proliferation of healthy and/or cancer cells).

According to a first embodiment, a combination of two types of radionuclides that have the advantage of both emitting the same type of radiation, namely an X-ray radiation, but present relatively different life times, is considered.

The latter combination is particularly interesting in case of large target volumes that are infected by cancer cells of variable aggressiveness. By way of example, the combination of ¹⁰³Pd (16.991 days) and ¹²⁵I (59.40 days) can be mentioned.

Another embodiment aims at combining various types of radionuclides that have the same kind of radiation, but with relatively different energy levels, in order to increase the efficiency of a treatment of heterogeneous tumours having an important volume.

By way of example, the association of ¹⁰³Pd (XR: 20-22 keV)+¹⁸¹W (XR: 60-70 keV) can be mentioned.

Following another embodiment, the association of two types of radionuclides that have very different types of radiation can be considered, in order to increase the efficiency of the treatment of heterogeneous areas by a “cross-fire” effect.

By way of example, the following can be mentioned:

³²P (Beta: 1.71 MeV)+¹⁰³Pd (XR: 20-22 keV);

⁹⁰Y (β, 2.27 MeV max)+¹⁰³Pd (XR: 20-22 keV);

⁹⁰Y (β, 2.27 MeV max)+¹²⁵I (XR: 60-70 keV);

²¹¹At (Alpha: 5.87 MeV)+¹⁰³Pd (XR: 20-22 keV);

²¹¹At (Alpha: 5.87 MeV)+⁹⁰Y (β, 2.27 MeV max).

According to another embodiment, at least one type of radionuclide is considered being combined with another element, which ensures localisation or diagnostic functions, within one and the same element for brachytherapy.

By way of example, elements used for the localisation of cancer cells by PET (Positron Emission Tomography) or SPECT (Single Photon Emission Tomography) can be mentioned. ¹⁸F (PET), ⁸⁹Zr (PET), ^{99m}Tc (SPECT), ¹¹¹In (SPECT) can be mentioned as examples.

### Preparation of the Source

The association of two types of radionuclides within one composite radioactive source can be carried out in two different ways: either two radionuclides that are already in radioactive form before being mixed can be associated, or two composites that, after radiation, will generate two radionuclides in order to create the radioactive source can be associated.

In the first case, the radionuclides are obtained individually, either directly through production in a nuclear reactor, for instance, by neutron activation, or starting from a production process by means of a particle accelerator. Once prepared and extracted by the adequate physical and chemical methods, these two types of radionuclides are mixed and if possibly linked to a support in order to create the composite source.

For example, they can be mixed by absorption within a resin, for instance an ion exchange resin.

According to another embodiment, the radionuclides can be prepared and mixed; so as to obtain a liquid that will be dried in order to obtain an ink that functions as composite source, such as disclosed in publication EP 1 082 729.

According to another embodiment, each radionuclide can be considered to be provided in the form of a preparation of dried ink, and then mixed with one another, at the end of their preparation, in order to obtain the composite radioactive source.

Another way of preparing the source is to deposit successive layers of each type of radionuclides that have been prepared individually, or even a layer made of a mixture of two radionuclides on an adequate support (adequate solid pharmaceutical carrier of the product of the invention) by means of physical and chemical deposit procedures.

Physical deposit procedures are procedures such as “ion plating”, “sputtering magnetron”, “evaporation”, CVD (“chemical vapour deposition”).

Other chemical methods, such electrolyte deposits, immersion in chemical solutions, ink impressions can also be considered.

According to another embodiment, either the mixture or any one of both radionuclides can be inserted in a biocompatible capsule (forming the adequate solid pharmaceutical carrier of the product of the invention).

Another alternative to realise the radioactive source according to the present invention aims at mixing, before irradiation, two non-radioactive composites that will generate the two types of radionuclides after irradiation.

In the latter case, the mixture is carried out before the irradiation, in any conventional way, such as the mixture of two powders, the creation of alloys or the deposit of successive layers on an adequate support, in order to obtain a composite material to be irradiated.

This composite material will be activated by irradiation with a particle beam coming from an accelerator or in a nuclear reactor. The final product will constitute the composite radioactive source.

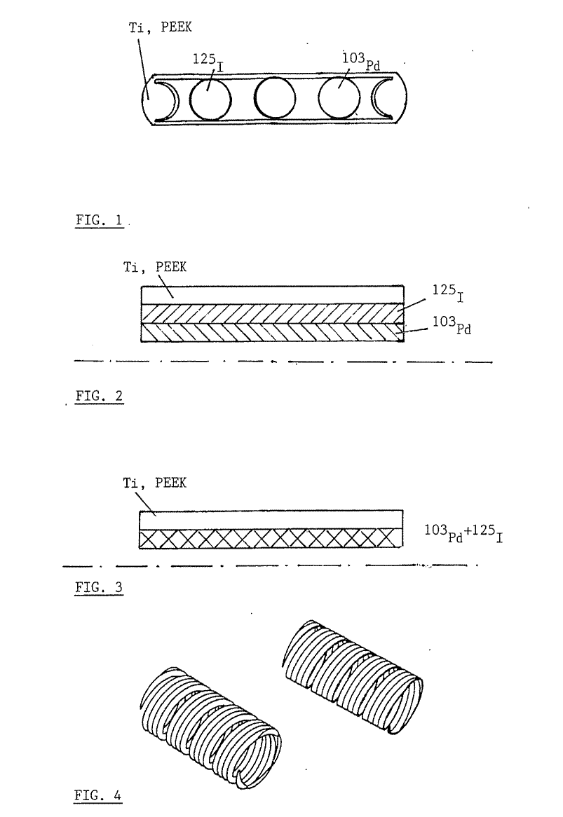

Examples of brachytherapy are presented in FIGS. 1 to 4.

In a first embodiment the composite radioactive source is included in a biocompatible capsule or envelope (forming the adequate solid pharmaceutical carrier of the product of the invention), as presented in FIG. 1.

By way of example, such capsules made of metallic materials (e.g. Ti) or polymeric materials (e.g. PEEK) are described in detail in the documents U.S. Pat. No. 1,753,287, U.S. Pat. No. 3,351,049 or U.S. Pat. No. 4,702,228.

If the procedure of successive deposits of two types of radionuclides (FIG. 2) or a mixture of both (FIG. 3) in layers is chosen, it is suitable to provide one last layer that covers the complete deposited material and to ensure that it is waterproof. Again, the nature of this material must be chosen in such a way that it is biocompatible (to form the adequate solid pharmaceutical carrier of the product of the invention). The same examples as the ones mentioned regarding forming a capsule or envelope can be used.

Finally, as presented in FIG. 4, the elements for brachytherapy (implants) can also appear in the form of a winding or a spring made starting from a wire in an alloy, which must also be biocompatible (to form the adequate solid pharmaceutical carrier of the product of the invention).

Preferred example: mixture of ¹⁰³Pd and ¹²⁵I within the same composite radioactive source for the treatment of prostate or breast cancer by brachytherapy.

Obtaining ¹⁰³Pd: ¹⁰³Pd can be obtained through two reactions, namely directly:
starting from ¹⁰²Pd, within a nuclear reactor through neutron irradiation by means of the reaction: (¹⁰²Pd (n, γ) ¹⁰³Pd)
starting from ¹⁰³Rh, using a loaded particle beam originating from an accelerator by means of the reaction: (¹⁰³Rh (p, n) ¹⁰³Pd).

The second method presents an advantage in relation to the first. Indeed, in the case of the first reaction, the ¹⁰²Pd enriched target also contains impurities that can be activated. The physical or chemical purification will therefore only affect these impurities and will not on the separation of ¹⁰³Pd from the other (radio) isotopes of the palladium. In the case of the second reaction, none of the other (radio) isotopes of the palladium is present in the target and a considerable purity of ¹⁰³Pd can be obtained through the chemical or physical separation of ¹⁰³Rh and ¹⁰³Pd. The specific activity of ¹⁰³Pd will thus be higher when using the second method. The half-life of ¹⁰³Pd will take 16.991 days and is therefore considered adequate for the treatment of aggressive tumours. However, due to its relatively low energy regarding the emission of X-rays (E_{average}=20.74 keV), it is necessary to implant a large number of implants in the tumour, which can reach to over one-hundred.

Obtaining ¹²⁵I: ¹²⁵I is quite easily obtained starting directly from nuclear reactions generated within a reactor. Its half-life is longer than the one of ¹⁰³Pd (59.40 days), which renders it adequate for the treatment of less aggressive tumours. Its XR radiation, on the contrary, presents a higher energy level (E_{average}=28.37 keV), and thus less implants required than compared to implants with ¹⁰³Pd.

One implant is particularly described in a patent application in the name of International Brachytherapy, Inc. (IBt in Belgium). This implant is made from two concentric tubes in titan.

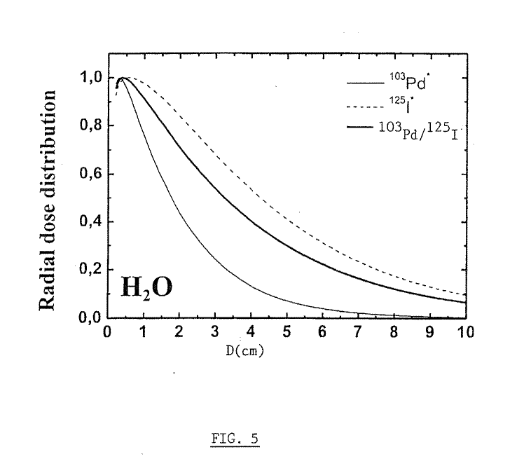

The present invention aims at making a mixture of ¹⁰³Pd-¹²⁵I taking the form of an ink, distributed homogenously in three bands printed on the inner tube, and for which ¹⁰³Pd is involved to an extent of 75% and ¹²⁵I to an extent of 25% of the total activity. A dose distribution as is presented in FIG. 5 will be obtained. This figure demonstrates that the axe of the abscissa represents the variation according to the distance of the dose deposited by the source during its life, multiplied by the square of the distance in order to leave out the decrease due to the solid angle and normalised by its maximum value.

As presented in FIG. 5, it is observed that, according to a ratio of the internal activities of ¹⁰³Pd/¹²⁵I, the distribution of the doses will vary in an interval between the curve corresponding to 100% of internal activity of ¹⁰³Pd and the curve of pure ¹²⁵I. It is therefore considered that, depending on the tumour, an optimal ¹⁰³Pd/¹²⁵I ratio can and will be selected.

From a temporal point of view, it is observed that the difference in half-life will induce a dose distribution that will evolve with time. Indeed, at the start of the irradiation of the tissues, the deposited dose will mainly be deposited on short distance due to the presence of ¹⁰³Pd. After several periods corresponding to the half life of ¹⁰³Pd, it will be observed that the deposited dose corresponds mainly to the presence of ¹²⁵I.

From a therapeutic point of view, it is noted that the main inconvenience of the use of ¹²⁵I (alone) in brachytherapy, lies in the fact that there is a non-zero irradiation of healthy cells that leads to an important morbidity, is eliminated, if not at least decreased. The fact of using a composite source of ¹⁰³Pd and ¹²⁵I will reduce the delivered dose on a long distance in a particularly advantageous way and will therefore noticeably reduce the risks of observable complications for the patient.

1. A radioactive implant for brachytherapy comprising a composite radioactive source comprising at least two types of different radionuclides.

2. The radioactive implant according to claim 1, wherein the two types of radionuclides, which are both defined by an atomic mass (A) and a number of protons, present either a different Atomic Mass or a different number of protons.

3. The implant according to claim 2, wherein the two types of radionuclides present a different number of protons.

4. The implant according to claim 1, wherein the two types of radionuclides present different characteristic radiations.

5. The implant according to claim 1, wherein the types of radionuclides present a same type of radiation but with different energy levels.

6. The implant according to claim 1, wherein the types of radionuclides present a same radiation but different half-life times.

7. The implant according to claim 1, wherein in addition to the types of radionuclides, the source further comprises contrast agents for imaging.

8. The implant according to claim 1, wherein the two radionuclides of two different types are present in such a ratio that the internal activity fractions are comprised within a range between 0.01/99.99% to 99.99/0.01%.

9. The implant according to claim 1, comprising either ¹⁰³Pd and ¹²⁵I or ¹⁰³Pd and ¹⁸¹W or ¹⁰³Pd and ¹³¹Cs.

10. The implant according to claim 1, comprising ¹⁰³Pd and ¹²⁵I, for which the fractions of internal activity are comprised within a ratio close to 75/25%.

11. A method for the treatment or prevention of local tumours in a patient such as breast tumours, prostate tumours, liver tumours, brain tumours, wherein the implant according to claim 1 is administered to the tumour or in the vicinity of the tumour of said patient.

12. The method according to claim 11, wherein the tumour is a local tumour selected from the group consisting of prostate tumours, breast tumours, liver tumours or brain tumours.

13. A method for eliminating stenosis or necroses of cells and/or tissues of a patient, wherein the implant according to claim 1 is administered in said tissue or in the vicinity of said tissue and/or said cells of said patient.

14. The method according to claim 13, wherein the implant is administered to the coronary arteries of the patient.

15. Implant according to claim 1, wherein the types of radionuclides are selected from the group consisting of the following elements: ¹⁴C, ³²P, ³³P, ³⁵S, ³⁶Cl, ⁵¹Cr, ⁵⁵Co, ⁶⁰Co, ⁶³Ni, ⁶⁴Cu, ⁶⁷Cu, ⁶⁸Ge, ⁹⁰Y, ⁸⁹Zr, ⁹⁹Mo, ^{99/99m}Tc, ¹⁰³Pd, ¹¹²Pd, ¹¹⁰Ag, ¹¹²Ag, ¹¹³Ag, ¹¹¹In, ¹²³I, ¹²⁴I, ¹²⁵I, ¹³¹I, ¹³³Xe, ¹³¹Cs, ¹³⁷Cs, ¹⁴² Pm, ¹⁵³Gd, ¹⁵⁹Gd, ¹⁶⁶Ho, ¹⁶⁹Yb, ¹⁸¹W, ¹⁸⁶Re, ¹⁸⁸Re, ¹⁹²Ir, ¹⁹⁴Ir, ¹⁹⁸Au, ¹⁹⁹Au, ²¹⁶Bi, ²¹¹At, ²⁴¹Am or other equivalent radioactive element.
